(19) United States
(12) Patent Application Publication
(10) Pub. No.: US 2016/0262677 A1
(43) Pub. Date: Sep. 15, 2016

(54) REMOVAL UNIT FOR BODILY FLUIDS, IN PARTICULAR BLOOD

(71) Applicant: GREINER BIO-ONE GMBH, Kremsmuenster (AT)

(72) Inventors: Franz EBETSBERGER, Kremsmuenster (AT); Gerhard STRASSER, Kremsmuenster (AT)

(73) Assignee: Greiner Bio-One GmbH, Kremsmuenster (AT)

(21) Appl. No.: 15/022,701

(22) PCT Filed: Sep. 15, 2014
(86) PCT No.: PCT/AT2014/050204

§ 371 Date: May 24, 2016

(30) Foreign Application Priority Data
Sep. 17, 2013 (AT) A 50588/2013

(51) Int. Cl. A61B 5/15

(52) U.S. Cl. CPC A61B 5/150213; A61B 5/150251; A61B 5/150343; A61B 5/150763

(57) ABSTRACT

The invention relates to a medical removal unit (1) for blood, comprising a needle carrier (2), at least one cannula (3), which has a distal and a proximal cannula segment (6, 7), and a hose-shaped sleeve (4), which has a closed sleeve end (12) and an open sleeve end (11). The sleeve (4) is fastened to a proximal needle-carrier end (10) by means of the open sleeve end (11) and surrounds the proximal cannula segment (7) in a sealing manner in such a way that an accommodating chamber (13) is formed. An air removal device (14) connects the accommodating chamber (13) to the outer environment in order that air flows out of the accommodating chamber and is formed by at least one outflow channel (15) arranged or formed in the needle carrier (2). A partial segment of the outflow channel (15) comprises a plurality of chambers (27, 28, 29) arranged one after the other and spaced apart from each other in an outflow direction and at least one connecting channel (30, 31) that connects chambers (27, 28, 29) arranged immediately adjacent in the outflow direction. The connecting channel has a flow cross-section that allows the air to flow through but at least largely prevents or completely stops the bodily fluid, in particular blood, from flowing through.

The invention relates to a medical removal unit for bodily fluids, in particular blood, as described in claim 1.

A generic blood sample taking needle arrangement is known from EP 0 619 096 B1 or U.S. Pat. No. 5,520,193 A. The latter comprises a needle cannula with a first end which is suitable for inserting into a vein of a blood donor. A second, obliquely cut end of the needle cannula is suitable for inserting into a blood collecting container under negative pressure, which can be connected in a scaling manner to the needle cannula. A needle cannula support device is attached in a middle section of the needle cannula so that the first end of the needle cannula extends outwards and is connected in a fluid-tight manner to a first side of the support device. The second end of the needle cannula extends outwards in an opposite direction thereto. Furthermore, an elastically resilient cap is attached on the support device over the second end of the needle cannula. The material for forming the support device is made from a see-through or transparent material. In the support device a radial passage is provided as an indication chamber, which is in flow connection via a passage extending in axial direction to the receiving chamber surrounded by the resilient cap. The radial passage is closed off from the external environment by an air-permeable, but liquid-rejecting stopper.

Another blood taking device is known from DE 32 29 783 C1 and comprises a double-ended cannula, to which a cannula holder is secured. At the proximal end of the cannula holder a tubular shoulder is arranged on which a flexible sleeve is held. Furthermore, on the tubular shoulder a porous ring is also provided. On the outside of the tubular shoulder in addition several longitudinal grooves are formed which extend at least up to the porous ring. The receiving chamber surrounded by the sleeve is in flow connection via the longitudinal grooves and the porous ring with the outer environment. The porous ring allows the outlet of air from the sleeve to the environment, but blocks the passage of blood. To display the success of the piercing the sleeve is made from a transparent latex.

A further display device for showing the success of the piercing of a blood taking device is known from U.S. Pat. No. 6,905,483 B2. The medical device comprises a needle carrier, in which an indication chamber is arranged which is sealed with a venting stopper from the external environment. The venting stopper is made from a material which enables the throughflow of air but prevents the throughflow of fluid. For this the closing stopper can comprise a membrane which has a plurality of microscopic openings or pores. A cannula at its proximal and distal end projects on both sides out of the needle carrier and is connected to the latter. A through channel of the cannula is in flow connection with the indication chamber. A hose-like sleeve surrounds the proximal cannula segment forming a receiving chamber. The blood flowing in on piercing from the distal end of the cannula displaces the air out of the cannula and pushes the latter via the indication chamber and the air-permeable closing stopper to the outer environment.

Additional blood taking needle arrangements comprising a filter element which enables the throughflow of air but prevents the throughflow of blood are known for example from EP 0 451 040 A1 and JP 36 74946 B2.

From EP 0 396 537 B1 and DE 37 85 363 T2 a device is known for displaying the flow which is used mainly for supporting and precisely positioning a catheter structure in a blood vessel. When in use for the clean placing of the catheter structure firstly a sharpened tip of the supporting needle or cannula to which the catheter structure is secured penetrates the blood vessel intended for this. When the sharpened tip is positioned correctly, blood flows continually through the hollow support needle from the tip to its proximal end, which is attached securely to a holding base. In order when setting the catheter structure to display to the medical staff the correct position of the catheter inside the blood vessel, the device for displaying the flow comprises a main body with an elongated, essentially cylindrical configuration, on the distal end of which a needle extends outwardly. The needle has a hollow inner section which extends along its length from the tip to its proximal end. The proximal end is secured to the inside of the main body, wherein the blood flows in along the needle into the base when the tip is placed in the blood vessel. Furthermore, on the outside of the main body an at least partly transparent casing device is arranged. On the outside of the main body also a flow channel is provided which is in the form of a groove structure with a plurality of groove segments, which are in fluid communication with one another by offset arranged connecting openings. At the end of the flow channel a venting opening is provided through which the air contained in the flow channel can flow out into the environment. By means of this winding designed flow channel during the setting of the catheter arrangement the correct positioning thereof inside the blood vessel can always be controlled. As soon as the catheter arrangement is set correctly, the device for displaying the flow is removed and the catheter arrangement remains on the patient.

The underlying objective of the present invention is to create a medical removal unit or piercing unit for the removal of bodily fluids, in particular blood, in which the success of the piercing is displayed automatically and in this case inexpensively without using a filter element and has a high degree of operational security.

Said objective of the invention is achieved by the features of claim 1.

The advantage of the features of claim 1 is that with the removal unit according to the invention it is possible during the piercing process for the air inside the cannula and the hose-like sleeve to flow out of the removal unit. This takes place through the outflow channel, which comprises a plurality of connecting channels arranged one behind the other in flow direction, in which the flow cross-section is selected such that although the passage of air is possible the passage of blood is prevented. By means of the additional provision of a plurality of chambers between the connecting channels arranged in the outflow channel in this way a kind of labyrinth seal can be created in which the sealing effect is achieved on a purely mechanical basis. By means of the labyrinthine arrangement of separate chambers one behind the another and connecting channels, thus inside the outflow channel for the amount of air to be removed different flow speeds can be achieved. By providing the purely mechanical air removal device the use of expensive filter elements can be avoided and despite this an outflow of the air provided in the cannula can be achieved during the piercing process. Thus afterwards not only can the piercing success be shown optically, but the outlet of the entering bodily fluids, in particular blood, can also be prevented by the air removal device. If in a repeated removal process and the associated, increased build-up of pressure inside the sleeve the amount of bodily fluid stored there, in particular blood, should pass through one of the connecting channels into the following chambers, this is used as an additional storage space for holding the bodily fluid. In this way the escape of the latter at the end of the outflow channel can be reliably prevented.

A further embodiment as claimed in claim 2 is also advantageous, as thus by means of the mutual displacement of connecting channels arranged directly behind one another in outflow direction in circumferential direction relative to one another there is diversion of the air flow inside the chamber arranged in between. Furthermore, however by means of the deliberate displacement with a possible inflow of bodily fluid, in particular blood, the further flow of air is possible. A direct further flow of the entering bodily fluid out of the chamber can then be reliably prevented up to a certain filling level thereof.

An embodiment as claimed in claim 3 is also advantageous, as a continuous connecting channel for forming the outflow channel is avoided. In this way not only can a labyrinth seal be created, but the unwanted outlet of bodily fluid from the outflow channel to the environment can be prevented. In this way in a simple manner it is possible for the air in the removal unit to flow out but despite this a high degree of operating safety is ensured.

By means of the embodiment as claimed in claim 4 it is possible to form the needle carrier on the basis of its physical division into the main body and the insert inserted and mounted therein to provide a structural unit which is simple to produce. In this way without additional, complicated production processes, in a simple manner a needle carrier can be produced, for example made from a plastic material, which can be produced with high precision in an injection molding die. In this way the chambers and the connecting channels can either be formed directly during the production process or in a simple post-processing step. The chambers can either be designed by coordinating the two components geometrically with one another or by a suitable choice of shape for the latter.

According to another embodiment variant according to claim 5 in this way a structural unit is created which is simple to produce which consists of components that can be joined together and in this way inexpensive manufacturing is possible.

A development as claimed in claim 6 is also possible as in this way a base part or main body can be created which is simple to manufacture in which a fully formed insert has to be simply inserted and fixed on the latter. Furthermore, in this way also the size and position of the individual chambers can be determined exactly.

According to one embodiment, as described in claim 7, annular chambers can be created simply which are simple to form and by means of a suitable arrangement and design of the connecting channels an outflow channel can be created which enables the unhindered outflow of air, but reliably prevents the throughflow of blood up to the outlet to the outer environment.

A development as claimed in claim 8 is also advantageous as thus the outflow channel in the insert can be designed in the form of thread, which is divided simply by several separating webs arranged in the longitudinal extension of the outflow channel into the individual chambers. In the region of the separating webs the corresponding connecting channels are arranged or formed so as to only enable the throughflow of air in said sections. By means of this spiral-shaped longitudinal route thus simply a plurality of chambers can be formed one behind the other, wherein this is dependent on the number and arrangement of the separating webs.

In the configuration as claimed in claim 9 or 10 it is an advantage that thus with an increasing flow length inside the removal unit the size and the associated receiving volume of the individual chambers can be increased in the direction of the outflow direction. Thus then also the security of the whole removal unit can be increased by the increasing and available receiving volume of the individual chambers.

An embodiment as claimed in claim 11 is also advantageous as thus a relatively large receiving volume can be obtained inside the individual chambers. Furthermore, also a flow is possible between the connecting channels on both sides in circumferential direction inside the respective channel.

By means of the embodiment as claimed in claim 12 a reliable, gas-tight mount for the cannula inside the needle carrier can be created. This connection could be formed either during the production process of the needle carrier, in particular its main body, but also in a subsequent connecting process, such as for example an adhesive process.

According to one embodiment, as described in claim 13, directly after the receiving space formed by the sleeve a directed throughflow of air to flow out can take place in the innermost part of the insert between the latter and the cannula. In this way there can be a linear outflow of air without large diversions, whereby the automatic outflow of air can be performed automatically by the relatively low blood pressure. In this way obstacles to the flow, which may otherwise be formed by diverting the airflow, are avoided.

In this case a configuration as claimed in claim 14 has proved advantageous because in this way during the piercing process the bodily fluid, in particular the blood, can flow automatically into the section of the outflow channel provided for this purpose and thus without additional means the correct positioning of the cannula inside the body of the patient can be made visible to the user.

According to an advantageous development as claimed in claim 15 thus a visual check of the piercing process can be performed at the start of the outflow channel inside the needle carrier. The air removal device can be arranged after the indication chamber in order in this way, at least when providing indication chambers, to enable the secure inflow of bodily fluid to that point. This flowing into the indication chambers is performed automatically during a correct piercing process, wherein up to the display the outflow of air from the indication chamber is performed through the outflow channel and the adjoining air removal device.

However, an embodiment as claimed in claim 16 is also advantageous, as thus it is possible to avoid having a separate indication chamber and directly after the outlet of bodily fluid in the region of the proximal end of the cannula the optical display can be provided simply in the region of the sleeve.

According to claim 17 by providing an indication chamber it is also possible to visually check for a correct piercing process in this area.

In the embodiment according to claim 18 thus a one-piece cannula can be used, in which between the distal and the proximal end a continuous, uninterrupted removal channel is provided with the same flow cross-section as a connection between the patient and the blood taking device. By means of this continuous, even flow cross-section negative influences on bodily fluids, in particular blood, can be achieved for a perfect subsequent analysis process.

An embodiment as claimed in claim 19 is also possible as thus the removal unit can be prefabricated as an assembly and can be inserted as necessary in a handling device provided for this purpose and can be connected thereto. In this way a simple, flexible use of the removal unit can be provided for a range of different conditions.

Lastly, however an embodiment is also possible as described in claim 20, as in this way a prefabricated assembly unit can be provided, with which it is possible to achieve a high degree of prefabrication and assembly. In this way without an additional assembly or joining process it is possible for the user to use the removal unit directly.

For a better understanding of the invention the latter is explained in more detail with reference to the following Figures.

In a much simplified, schematic view:

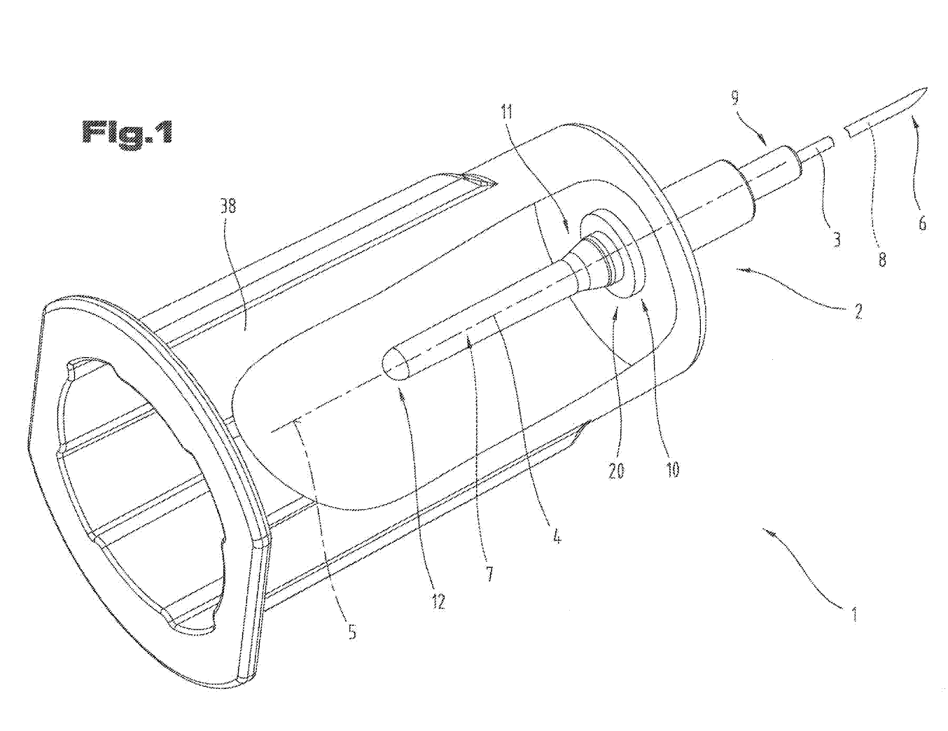

FIG. 1 shows a first embodiment of a removal unit with an additional handling element arranged thereon, in a diagrammatic view;

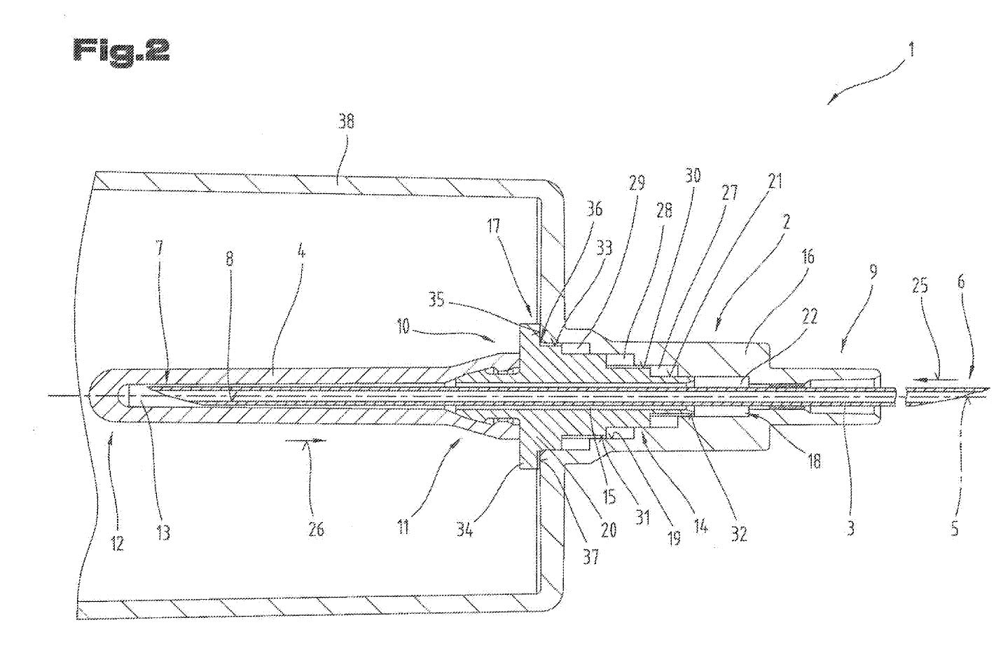

FIG. 2 shows the removal unit with the handling element according to FIG. 1, in axial cross-section;

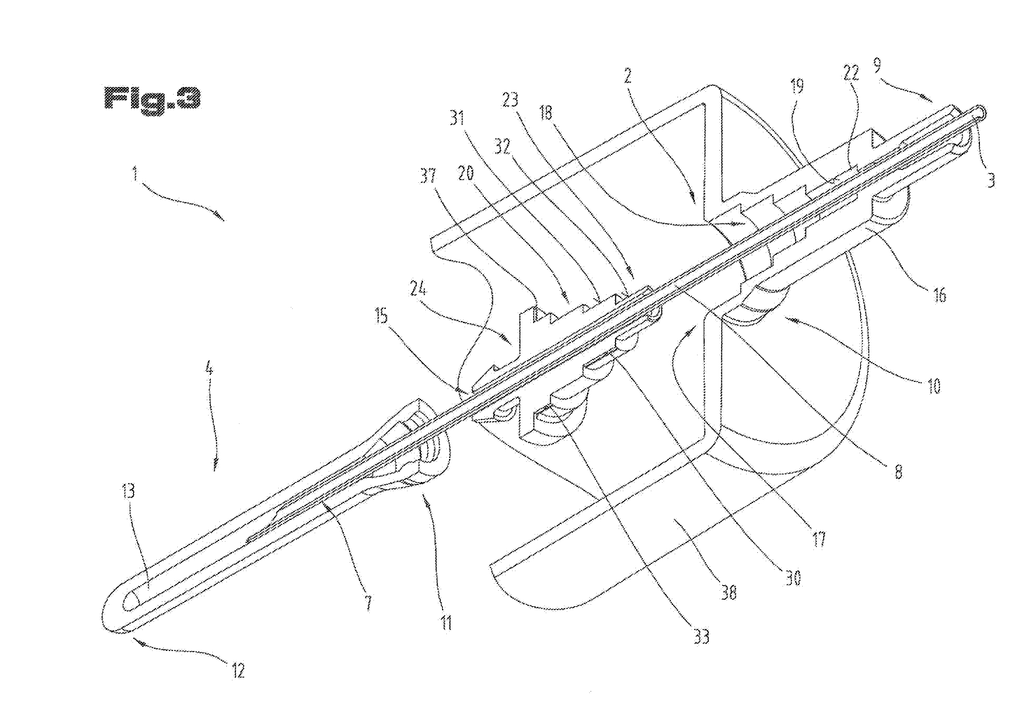

FIG. 3 shows the removal unit with the handling element according to FIGS. 1 and 2, in a position of the individual components spaced apart from one another, in axial cross-section and a diagrammatic view;

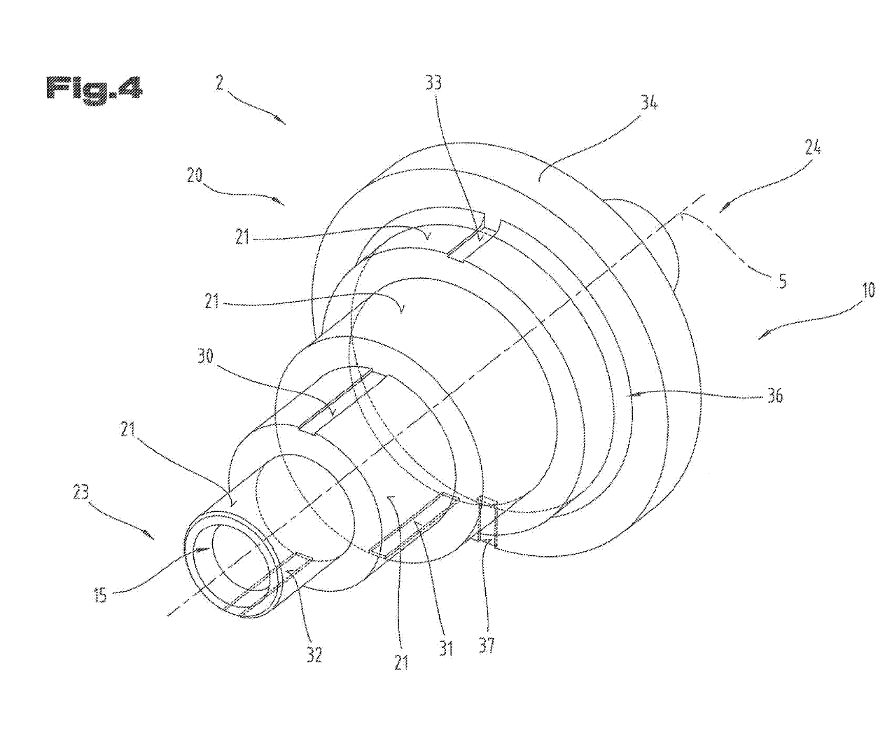

FIG. 4 shows the insert of the needle carrier of the removal unit according to FIG. 1 to 3, in a diagrammatic view;

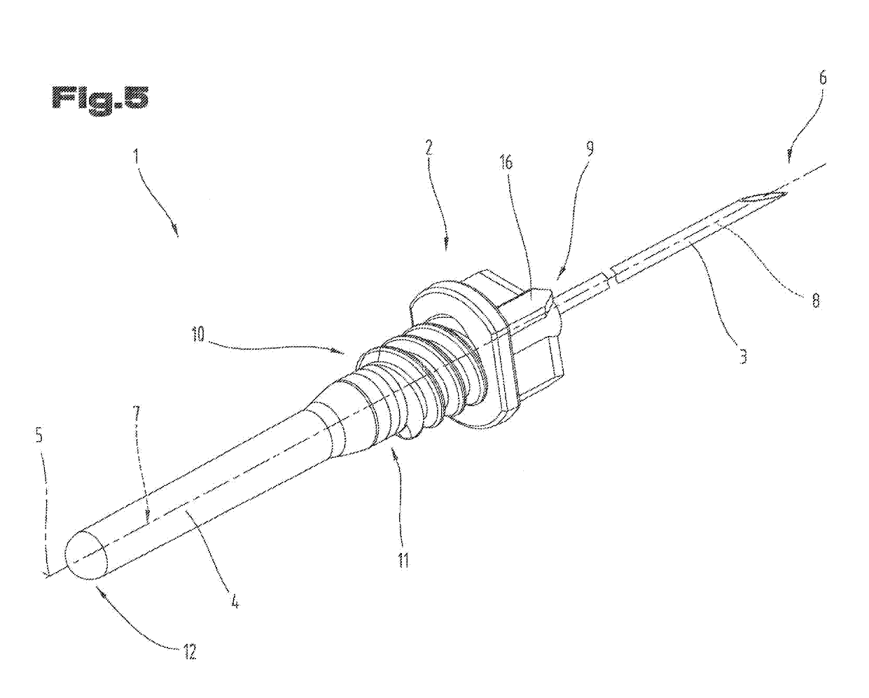

FIG. 5 shows a second embodiment of a removal unit, in diagrammatic view;

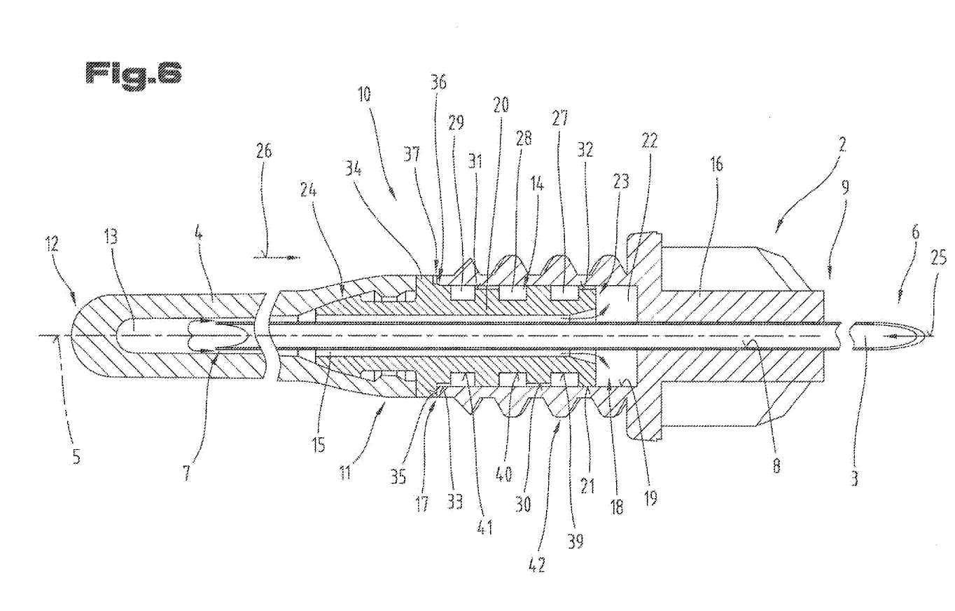

FIG. 6 shows the removal unit according to FIG. 5, in axial cross-section;

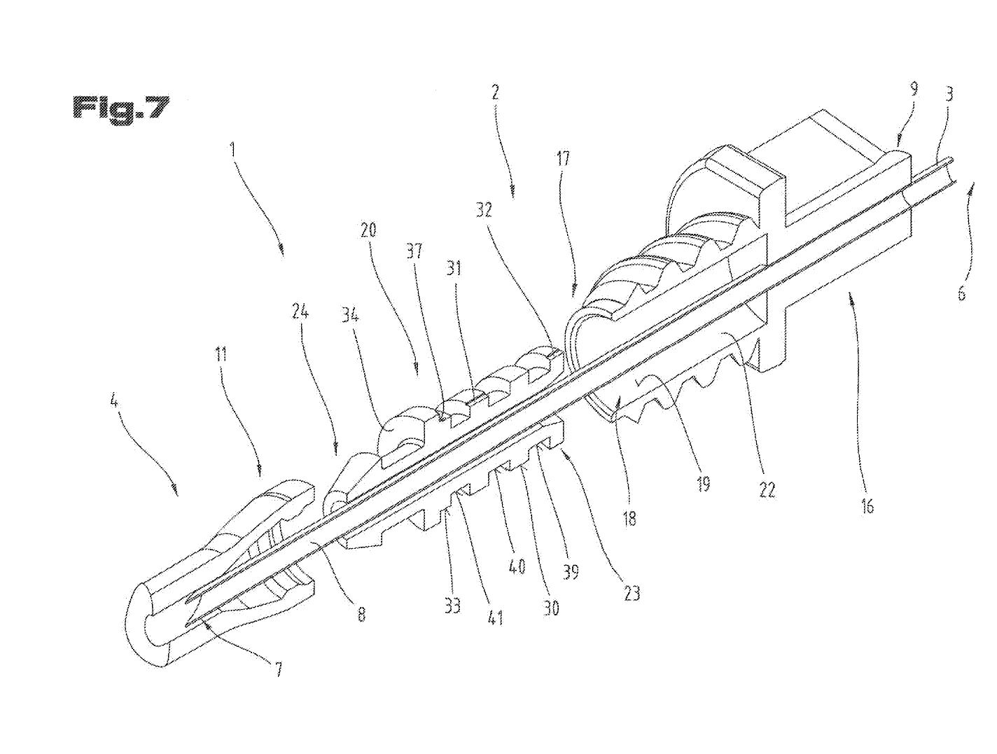

FIG. 7 shows the removal unit according to FIGS. 5 and 6, in a position of the individual components spaced apart from one another in axial cross-section and in a diagrammatic view;

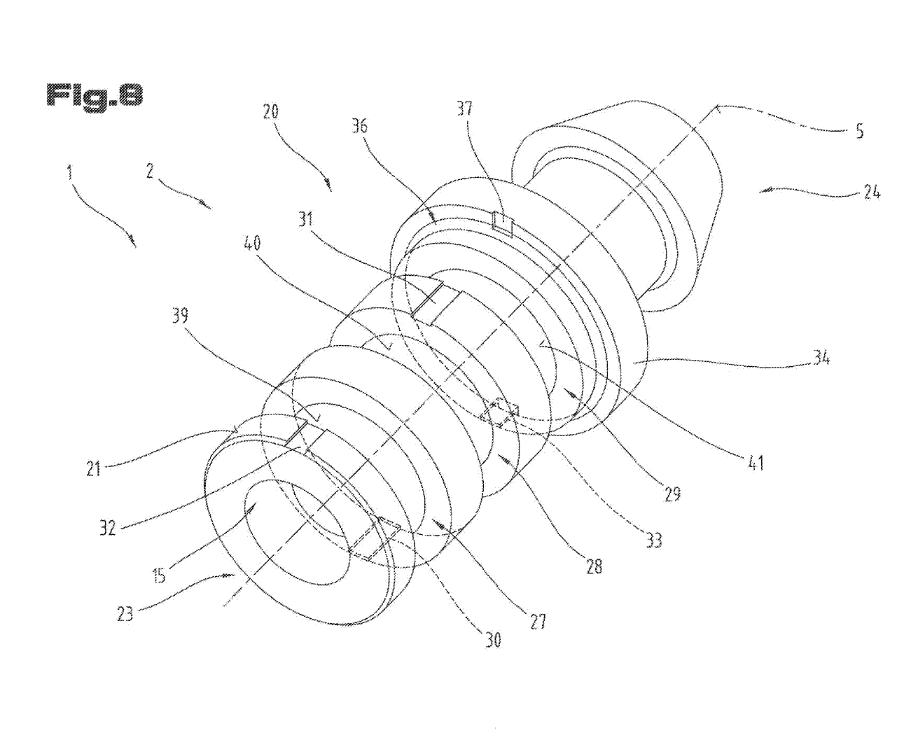

FIG. 8 shows the insert of the needle carrier of the removal unit according to FIG. 5 to 7, in a diagrammatic view;

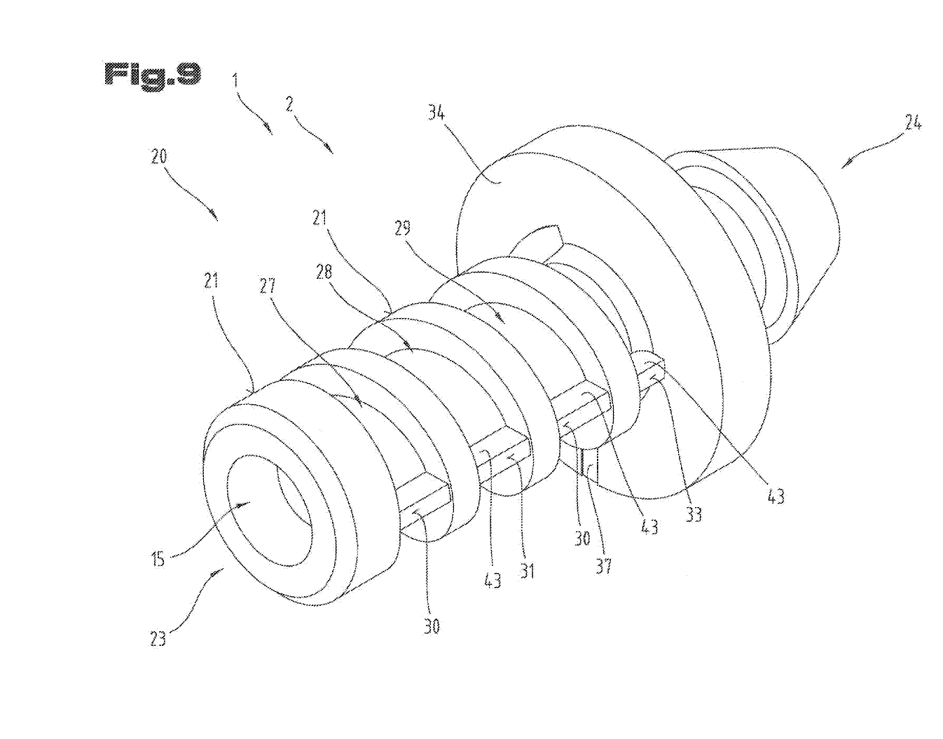

FIG. 9 shows a third embodiment of a removal unit, in diagrammatic view;

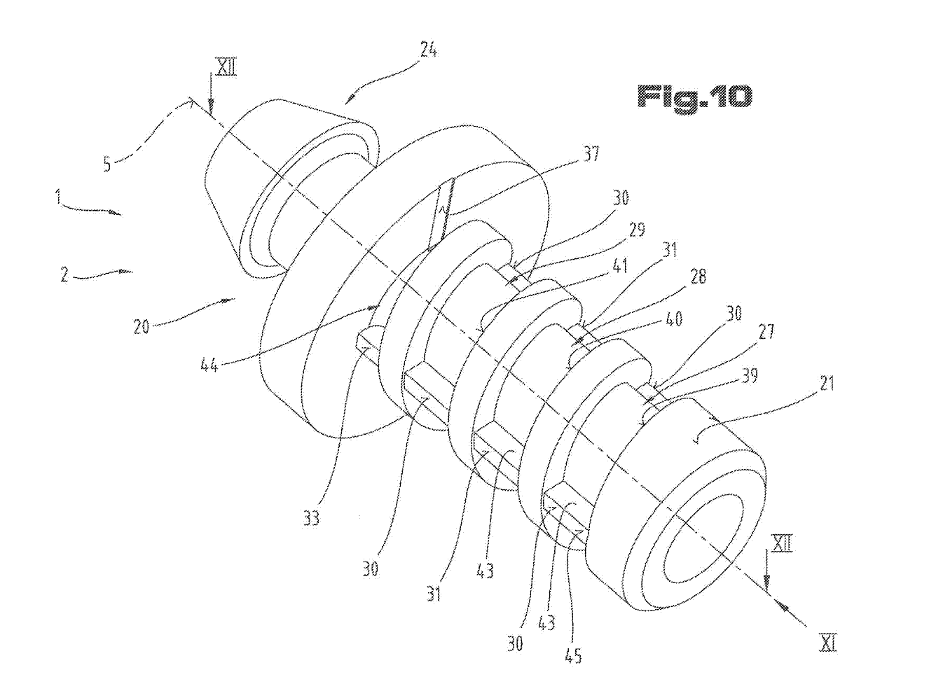

FIG. 10 shows the removal unit according to FIG. 9, in a different diagrammatic view;

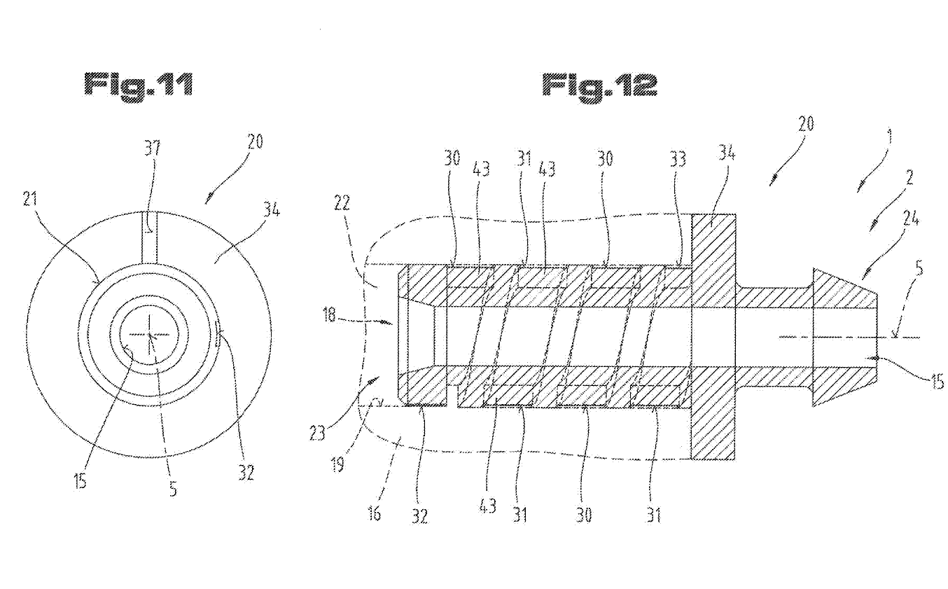

FIG. 11 shows the removal unit according to FIGS. 9 and 10, in a view according to arrow XI in FIG. 10;

FIG. 12 shows the removal unit according to FIG. 9 to 11, in axial cross-section according to the lines XII-XII in FIG. 10.

First of all, it should be noted that in the variously described exemplary embodiments the same parts have been given the same reference numerals and the same component names, whereby the disclosures contained throughout the entire description can be applied to the same parts with the same reference numerals and same component names. Also details relating to position used in the description, such as e.g. top, bottom, side etc. relate to the currently described and represented figure and in case of a change in position should be adjusted to the new position.

FIG. 1 to 4 show a first possible embodiment of a medical removal unit 1, which comprises at least one cannula 3 and a hose-like, elastically deformable and pierceable sleeve 4.

Furthermore, it should be mentioned here in general that in the present application the term “distal” denotes the part of the removal unit 1, which during the correct use by a user (medical staff) faces away from the latter and thus for example faces the body of a patient. The term “proximal” always denotes the part of the removal unit 1 which during the correct use by a user (medical staff) faces the latter and thus for example faces away from the body of the patient. However, the same also applies to the denotation of the direction of individual components forming the removal unit 1, which can have distal and proximal ends or sides respectively as viewed in the direction of a longitudinal axis 5, which in the following are denoted in more detail with the corresponding component name.

The removal unit 1, comprising the needle carrier 2 with the cannula 3 secured thereon, can also be denoted as a piercing unit, which can be used alone or can also be attached to a handling device described in more detail below or can be designed partly with the latter.

The removal units 1 described here are mainly used in the medical field for the removal of bodily fluids, in particular blood. For this a distal cannula segment 6 is pierced in a known manner by a user, such as for example a doctor or medical staff, into the body of the patient into an intended vein or artery. For the subsequent removal process mostly evacuated blood sample removal tubes with a proximal cannula segment 7 of the cannula 3 are moved into flow connection one after the other by a piercing process. Such blood sample taking tubes, which can also be referred to as collecting containers, have a penetrable, self-closing sealing body, in particular a sealing stopper, which can also be provided with a cap for simpler handling. To form the flow connection the sealing body or the sealing stopper of the blood sample taking tube is pierced by the proximal cannula segment 7 and thus a flow connection is formed between the vein or the artery of the patient and the inner chamber of the blood taking tube designed as a collecting container. During the piercing process the sleeve 4 covering the proximal cannula segment 7 is pierced by the cannula 3 and when pushing it in further the sleeve 4 is pushed in the direction of the needle carrier 2 and thereby folded.

This thus formed flow connection between the distal cannula segment 6 and the proximal cannula segment 7 can also be referred to as a so-called removal channel 8, which connects the distal cannula segment 6 to the proximal cannula segment 7 for throughflow. Preferably, the removal channel 8 is designed to be continuous between the distal cannula segment 6 and the proximal cannula segment 7 inside the cannula 3. In this embodiment it is also possible to refer to a double-ended, through cannula 3.

The needle carrier 2 can be formed by an assembly consisting of several components, wherein the needle carrier 2 has a distal needle carrier end 9 and a proximal needle carrier end 10.

The hose-like sleeve 4 comprises in turn an open distal sleeve end 11 and at the opposite end a closed proximal sleeve end 12. The open distal sleeve end 11 is secured onto the proximal needle carrier end 10 of the needle carrier 2. This can be performed by simply pushing the open distal sleeve end 11 over a shoulder with a retaining device on the needle carrier 2, as is generally and sufficiently known. In this way the sleeve 4 surrounds at least a portion of the cannula 3 in the region of its proximal cannula segment 7. By means of this complete enclosure an accommodating chamber 13 is formed inside the sleeve 4, which on the one hand is closed by the inner sleeve chamber and on the other hand is sealed by the holding shoulder of the needle carrier 2.

Furthermore, an air removal device 14 is provided inside the needle carrier 2, through which the air in the cannula 3 and in the accommodating chamber 13 can flow out to the outer environment. For this purpose in the needle carrier 2 an outflow channel 15 is also provided with preferably a plurality of portions, whereby in the present example embodiment the air removal device 14 forms a portion of the outflow channel 15.

The free, obliquely ground proximal end of the cannula 3 opens with its proximal cannula segment 7 into the accommodating chamber 13 of the sleeve 4. The inner surface of the sleeve 4 is designed to run at a radial distance relative to the outside of the cannula 3 in axial direction up to the distal sleeve end 11 in the connecting area with the needle carrier 2. In this way in an advantageous case without the inner sleeve wall fitting around the outside of the cannula 3 an annular channel is formed extending up to the needle carrier 2, which annular channel forms a first portion of the outflow channel 15.

As already described above, the needle carrier 2 can be formed by a component group consisting of several components. Although such medical removal units, which come into contact with blood, are disposable products for single use, the intention is to guarantee their reliable application with the lowest possible outlay for components and component costs. This is an essential consideration, in particular so as to avoid the contamination or the transmission of infections such as for example AIDS, hepatitis or the like.

In the present example embodiment the needle carrier 2 comprises a main body 16, which at its proximal end section 17 has a receiving space 18 extending into its interior. The receiving space 18 is delimited by an inner surface 19. Furthermore, the needle carrier 2 also comprises an insert 20 received in the receiving space 18 and held on the main body 16, which in turn comprises an outer surface 21 facing the inner surface 19.

A second section of the outflow channel 5 is then arranged or formed in outflow direction adjoining the accommodating chamber 13 and the intermediate space formed between the sleeve 4 and the cannula 3 between the outside of the cannula 3 and the insert 20 of the needle carrier 2. In this embodiment of the outflow channel 15 it is possible for example that the inner side of the insert 20 is arranged or formed spaced apart over the whole axial extension in radial direction from the outside of the cannula 3. In this way in this section a continuously formed annular channel is provided which enables the further flow of air from the proximal cannula segment 7 into the needle carrier 2.

Independently of this however it would also be possible to design the outflow channel 15 in the region of the insert 20 by a plurality of longitudinal grooves running offset to one another over the inner circumference of the insert 20, in order in this way to enable an at least partial bearing or support of the insert 20 on the cannula 3. In this case it is also possible to provide a certain mounting or securing of the insert 20 on the cannula 3. This could be achieved by means of a press-fit and/or an adhesive connection and/or a welding connection or the like.

Furthermore, inside the outflow channel 15 an indication chamber 22 can be provided or formed for displaying the success of the piercing for the subsequent removal of blood. The indication chamber 22 is provided upstream of the air removal device 14 in air flow direction.

The insert 20 comprises in axial direction—i.e. as viewed in the direction of the longitudinal axis 5—a distal end 23 and a proximal end 24. In this case the proximal end 24 of the insert 20 also forms the proximal needle carrier end 10.

By inserting the insert 20 into the receiving space 18 of the main body 16 the indication chamber 22 can be formed or delimited by a portion of the receiving space 18 of the main body 16 and the distal end 23 of the insert 20. Inside the removal channel 8 between the distal cannula segment 6 and the proximal cannula segment 7 a first flow direction is entered with an arrow 25. Said first flow direction according to arrow 25 is also the flow direction that the blood to be removed takes from the vein or artery of the patient to the blood removal tube. With the entry of bodily fluid, in particular blood, into the removal channel 8 the air in the latter is moved or pushed out of the removal channel 8 towards the proximal cannula segment 7 and on into the accommodating chamber 13.

As venous blood pressure is usually in a range with a lower limit of 3 mmHg (in millimeters mercury column) and an upper limit of 8 mmHg, it is necessary for the automatic flow movement inside the removal channel 8 that the volume of air present there is displaced. The blood pressure range within the set limits in a different measurement unit is e.g. 20 mbar (millibar) to 120 mbar. As the compression of air is not possible at such low pressures and in the small amount of space available inside the needle carrier 2 or accommodating chamber 13, the previously described outflow channel 15 is provided with different portions, in particular the air removal device 14.

In said outflow channel 15 in the present example embodiment passing from the accommodating chamber 13 through the insert 20 possibly into the indication chamber 22 there is an outflow of air in opposite flow direction, as indicated by an additional arrow 26 in the region of the outside of the sleeve 4. Furthermore, the outflow channel 15 should be dimensioned so that with an undeformed sleeve 4 and the successful piercing of the distal cannula segment 6 the bodily fluid to be taken from the patient, in particular blood, flows through the removal channel 8 at least up to the proximal cannula segment 7 in the first flow direction according to arrow 25 into the accommodating chamber 13. If for example the sleeve 4 itself or a portion thereof is made from a translucent or transparent material, here already the success of the piercing can be made optically visible. In this case the provision or arrangement of the indication chamber 22 could be omitted.

If this is not the case, the unhindered subsequent further flow of entering blood in the opposite flow direction according to arrow 26 into the indication chamber 22 should be made possible. It should be noted that the indication chamber 22 can also be arranged or provided in a position inside the outflow channel 15. In addition, it is advantageous if the needle carrier 2, in particular its main body 16, is made at least partly from a translucent or transparent material.

The cannula 3 is usually made from a metal material, in particular a rust-free, high-quality stainless steel material, in order on the one hand to keep the piercing load as low as possible and on the other hand to avoid the risk of infection.

By means of the preferred provision of a continuous, double-ended cannula 3 the removal channel 8 is designed to have a continuous even flow cross-section. This has the advantage that when the blood flows through during its removal it cannot be subjected to negative influences, such as for example hemolysis or the like. In this way the quality of the blood sample can be maintained or achieved without being affected by the removal process.

The air removal device 14, which is formed by at least the additional portion of the outflow channel 15 arranged or formed in the needle carrier 2, then extends inside the needle carrier 2 between the inner surface 19 delimiting the receiving space 18 and the outer surface 21 delimiting the main body 16. In this additional flow section of the outflow channel 15 there is a change in the flow direction. This additional flow direction is aligned in the present example embodiment in the same direction, as entered in the removal channel 8 in the first flow direction entered previously by the arrow 25. In this way the first and the last flow direction is aligned running from distal to proximal and the second flow direction is aligned running from proximal to distal.

Thus a further portion of the outflow channel 15 is formed by a plurality of chambers 27, 28, 29 spaced apart from one another and arranged one behind the another in outflow direction, wherein between the spaced apart chambers 27, 28, 29 at least one connecting channel 30, 31 extends connecting between chambers 27, 28; 28, 29 arranged directly adjacent to one another in outflow direction. The latter then together form the air removal device 14.

The connecting channel or channels 30, 31 here have a flow cross-section, which enables the throughflow of air, but prevents at least mostly or completely hinders the passage of bodily fluid, in particular blood. Preferably, chambers 27, 28, 29 arranged directly adjacent to one another in flow direction are arranged to be offset to one another in circumferential direction. In this case peripheral offsetting can be selected, e.g. diametrically to one another. It should be mentioned that the number of both the chambers 27 to 29 and connecting channels 30, 31 can be selected freely. In the present example embodiment three chambers 27 to 29 are shown, but it is possible however to select a larger or smaller number. The individual chambers 27 to 29 have a much larger volume or a much larger flow cross-section with respect to the connecting channels 30, 31. This is the case because with a repeated removal process with the same removal device 1 the blood provided at the beginning of the outflow channel 15 can be put under pressure by repeatedly inserting and piercing the blood sample removal tube on the cannula 3 in the outflow channel 15. Owing to this buildup of pressure and the thus produced overpressure a minimal amount can also be pushed in through one of the connecting channels 30, 31 in the outflow direction of air into one of the chambers 27 to 29.

As already described above, the individual chambers 27 to 29 are arranged or formed between the inner surface 19 of the receiving space 18 of the main body 16 and the outer surface 21 of the insert 20. Furthermore, it is the case in the example embodiment shown here that, as viewed in axial cross-section, the inner surface 19 of the receiving space 18 of the main body 16 is designed to widen step-like in the direction of its proximal end section 17. At the same time as viewed in axial cross-section however also an outer surface 21 of the insert 20 is designed to widen step-like in the direction of its proximal end 24. By means a suitable coordination of the dimensions with one another on the one hand the individual chambers 27, 28, 29 are formed and on the other hand there is a sealing bearing of the outer surface 21 of the insert 20 on the inner surface 19 of the main body 16. The flow connection between the thus formed chambers 27 to 29 is performed by the connecting channels 30, 31, which are preferably arranged or formed recessed in the outer surface 21 of the insert 20. Thus the connecting channels 30, 31 can be formed for example by longitudinal grooves with a very small flow cross-section in cooperation with the inner surface 19 of the receiving space 18. The chambers 27 to 29 can be designed to run respectively over the circumference. The latter can have a circular round form relative to the longitudinal axis 5, wherein also any other shape such as an oval, square, multi-sided or polygonal shape is possible.

In order to also enable the throughflow of air for example from the indication chamber 22 of the outflow channel 15 into the first chamber 27, a first additional connecting channel 32 can be provided, which is shown in an overview of FIGS. 2 and 3. The outflow of air from the last chamber 29 here in flow direction can be performed through a second additional connecting channel 33. Said additional connecting channel 33 ends at a ring base 34 formed in the region of the proximal end 24 of the insert 20. Said flange-like ring base 34 can be supported on an end wall 35 or end face of the main body 16. In this case the facing surfaces of the ring base 34 and the end wall 35 can be arranged in a plane aligned perpendicular to the longitudinal axis 5. By supporting the ring base 34 on the end wall 35 between the latter from the additional connecting channel 33 an additional section of the outflow channel 15 can extend, namely an annular channel 36. On a side opposite the additional connecting channel 33 an outflow channel 37 extending in radial direction can be arranged or formed. Through said outflow channel 37 the air flowing through the outflow channel 15 can exit to the outer environment.

In order to secure the cannula 3 onto the needle carrier 2 or its main body 16 on the cannula 3, preferably the main body 16 is connected securely to the cannula 3. This can be performed for example by means of a known adhesive process. In this way the cannula 3 is connected in this section in a sealing manner to the main body 16.

The previously described annular channel 36 used for the outflow of air from the outflow channel 15 can be formed by a corresponding clearance from the respectively facing corner areas between the main body 16 and the insert 20. The individual steps of the insert 20 forming the outer surfaces 21 are formed one after the other having an increasing radial distance from the proximal end 24. The outer surface 21 of the last step to which the ring base 34 connects directly can be formed in a corresponding manner in its corner area and thus the transition to the inner surface 19 of the receiving space 18 to the end wall 35 of the main body 16.

The throughflow cross section of the first additional connecting channel 32, which here in the present example embodiment is the indication chamber 22 with the first chamber 27 and the second additional connecting channel 33 connecting the last chamber 29 to the annular channel 36, can be selected in the same way as already described above for the connecting channels 30, 31. The previously described peripheral offsetting of the additional connecting channels 32, 33 can be performed relative to the first connecting channel 30, which connects the first chamber 27 to the second chamber 28, and relative to the second connecting channel 31, which connects together the second chamber 28 and the third chamber 29.

In this way, if the indication chamber 22 is provided, the outflow of air is performed between the main body 16 and the insert 20 such that the air can flow firstly via the first additional connecting channel 32 into the first chamber 27. The further flow of air from the first chamber 27 into the second chamber 28 is performed via the first connecting channel 30, which, offset peripherally, namely opposite the first additional connecting channels 32, is formed on the insert 20 as a longitudinal groove. The air that has entered into the second chamber 28 flows via the second connecting channel 31 into the third chamber 29, wherein this is performed on the opposite side relative to the first connecting channel 30. From the third and here last chamber 29 the outflow channel 15 continues in the second additional connecting channel 33 and opens into the annular channel 36. The outflow from the annular channel 36 to the outer environment is performed on the opposite side or relative to the second additional connecting channel 32 through the outflow channel 37 extending in radial direction.

By providing the individual connecting channels 30 to 33 with a very small flow cross-section and the chambers 27 to 29 in between with a much larger flow cross-section or holding volume a so-called labyrinth seal is formed. By means of the very small flow cross-sections of the connecting channels 30 to 33 this is designed to prevent blood from penetrating into this section of the outflow channel 15. However, if this still occurs for the aforementioned reasons during several consecutive removal processes, the individual chambers 27 to 29 are used as storage space in order to reliably prevent the outlet of blood from the outflow channel 15, in particular its outflow channel 37.

In order to make it difficult for blood to escape or flow into the chambers 27 to 29 or to completely prevent this, or prevent blood that has entered from flowing further, a substance could be introduced into the indication chamber 22 and/or at least one of the chambers 27 to 29 and/or at least one of the connecting channels 30 to 33, which is used for example to coagulate the blood that has entered and thus allow it to solidify or harden and thus achieve a certain stopper effect with the solidified blood or components thereof. In this way it is possible to achieve an even better sealing of the outflow channel 15 in this section. By selecting very small flow cross-sections in the region of the connecting channels 30 to 33 a deliberate stopping and associated closure of the outflow channel 15 could be achieved in this section. Thus the seal and thereby the outflow of blood from the outflow channel 15 to the environment could be prevented additionally. In this way contact with the removed bodily fluid is prevented and the risk of contamination is eliminated.

Independently of this it would also be possible however to use a component, which is not described in more detail, which receives the blood that has entered so far and visually indicates the success of the penetration, but prevents blood from flowing further. If blood has entered the indication chamber 22, so much air has already flowed out of the outflow channel 15 that from this point in time the section of the outflow channel 15, which extends between the inner surface 19 of the receiving space 18 and the outer surface 21 of the insert 20, is no longer absolutely necessary for additional outflow.

The removal unit 1 shown and described in this first embodiment comprises in the present embodiment an additional handling element 38, the needle carrier 2, in particular its main body 16, being connected to its distal face end. For this preferably a one-piece embodiment of the main body 16 and the handling element 38 is selected. In this way the proximal cannula segment 7 covered by the sleeve 4 projects into the handling element 38, as generally known.

This whole unit consisting of the handling element 38 and the removal unit 1 can be referred to as a handling component or handling unit for medical purposes. The handling element 38 comprises in a known manner a main body part, which comprises a distal end and a proximal end spaced apart in the direction of the longitudinal axis 5. In this case the proximal end is designed to be open and is used for receiving at least a portion of a blood removal device which is not described and shown in more detail, namely preferably an evacuated receiving container. In the region of the distal end a container wall is arranged which is aligned preferably in a plane perpendicular to the longitudinal axis 5, which is connected to the main body 16 preferably forming a unit.

FIG. 5 to 8 show a second and possibly independent embodiment of the removal unit 1, wherein for the same parts the same reference numerals and component names have been used as in the preceding FIG. 1 to 4. To avoid unnecessary repetition, reference is made to the detailed description in the preceding FIG. 1 to 4.

The embodiment of the removal unit 1 shown here is a prefabricated assembly of a cannula arrangement, which can be connected to a previously described handling element 38 or inserted therein.

The removal unit 1 shown here comprises the needle carrier 2 formed by the main body 16 and the insert 20, the at least one cannula 3 held therein or thereon, which covers the proximal cannula segment 7 thereof. In principle, the structure, in particular of the needle carrier 2 and the sleeve 4, corresponds to the one already described in detail for FIG. 1 to 4. To avoid unnecessary repetition therefore only the differences from the embodiment described above in FIG. 1 to 4 are discussed in detail.

The main body 16 comprises the receiving space 18 with its inner surface 19 delimiting the latter. Unlike the receiving space 18 described above for FIG. 1 to 4 said receiving space 18 has an approximately cylindrical continuous inner surface 19 spaced equally radially relative to the longitudinal axis 5. The insert 20 is inserted into said receiving space 18, wherein here too the outer surface 21 is also designed at least in the area opposite the receiving space 18 to be cylindrical relative to the longitudinal axis 5. For the embodiment of the previously described chambers 27 to 29 it is the case here that the latter are formed respectively by recesses 39 to 41 which are recessed in the outer surface 21 of the insert 20. Depending on the number of chambers 27 to 29 to be formed also a suitable number of recesses 39 to 41 need to be selected. If the recesses 39 to 41 for forming the individually separated chambers 27 to 29 are designed to be continuous over the periphery, the latter can be referred to as grooves or annular grooves. The individual recesses 39 to 41 are spaced apart from one another in the direction of the longitudinal axis 5 and are in flow connection respectively via the aforementioned connecting channels 30, 31.

The outflow channel 15 begins in the region of the accommodating chamber 13 surrounded by the sleeve 4 in the region of the proximal cannula segment 7 and extends here too between the outside of the cannula 3 and the inside of the insert 20 up to the indication chamber 22, if provided. The further flow path leads via the first additional connecting channel 32 into the first chamber 27 and from there via the first connecting channel 30 into the second chamber 28. The second chamber 28 is connected via the second connecting channel 31 to the third chamber 29. Lastly, the last and third chamber 29 are connected via the second additional connecting channel 33, the adjoining annular channel 36 and the radially aligned outflow channel 37 to the outer environment. The relative peripheral displacement of the individual connecting channels 30 to 33 can be performed in a similar manner, as has already been described in detail above.

To connect the needle carrier 2, in particular its main body 16, to a handling element 38, not shown in more detail, the latter is provided with an external thread 42. In this way the needle carrier 2 can be connected to a distal face end of a conventional handling element 38 or handling device, not shown in more detail, for receiving a blood sample tube, in particular screwed in. The external thread 42 can be a single or multiple thread arrangement, which can be selected as a function of the handling element 38 to be connected respectively.

If in this embodiment the indication chamber 22 is also provided, the needle carrier 2, in particular its main body 16, is made at least partly from a translucent or transparent material. However, it would also be possible for the sleeve 4 to be made at least partly from a translucent or transparent material.

The previously described annular channel 36 is used for the outflow of air from the outflow channel 15 and can be formed by a suitable clearance of the respectively facing corner areas between the main body 16 and the insert 20.

The flow cross-section of the first additional connecting channel 32, which is here in the present example embodiment the indication chamber 22 with the first chamber 27 and the second additional connecting channel 33 connecting the last chamber 29 to the annular channel 36, can be selected in the same way as already described above for the connecting channels 30, 31. The previously described peripheral offsetting of the additional connecting channels 32, 33 relative to the first connecting channel 30, which connects the first chamber 27 to the second chamber 28, and/or relative to the second connecting channel 31, which connects the second chamber 28 and the third chamber 29 to one another, can be performed in a similar way.

In this way if the indication chamber 22 is provided, the outflow of air between the main body 16 and the insert 20 is performed such that the air can flow firstly via the first additional connecting channel 32 into the first chamber 27. The further flow of air from the first chamber 27 into the second chamber 28 is performed via the first connecting channel 30, which is designed to be offset peripherally, namely opposite the first additional connecting channel 32, on the insert 20 as a longitudinal groove. The air entering into the second chamber 28 flows via the second connecting channel 31 into the third chamber 29, wherein this is performed in turn on the opposite side relative to the first connecting channel 30. From the third and here last chamber 29 the outflow channel 15 continues in the second additional connecting channel 33 and opens into the annular channel 36. The outflow from the annular channel 36 is performed on the opposite side or the second additional connecting channel 32 through the outflow channel 37 extending in radial direction to the outer environment.

By providing the individual connecting channels 30 to 33 with the very small flow cross-sections and the chambers 27 to 29 in between with a much larger flow cross-section or holding volume a so-called labyrinth seal is formed. The latter is designed to prevent by means of the very small flow cross-sections of the connecting channels 30 to 33 that blood can penetrate into this section of the outflow channel 15. However, if this should occur for the aforementioned reasons during a plurality of consecutive removal processes the individual chambers 27 to 29 are used as storage space in order to reliably prevent the outlet of blood from the outflow channel 15, in particular its outflow channel 37.

In order to make it difficult for blood to escape or flow into the chambers 27 to 29 or to completely prevent this, or prevent blood that has entered from flowing further, a substance could be introduced into the indication chamber 22 and/or at least one of the chambers 27 to 29, which is used for example to coagulate the blood that has entered and thus achieve a stopper effect with the solidified blood or components thereof. By selecting very small flow cross-sections in the region of the connecting channels 30 to 33 a deliberate stopping and associated closure of the outflow channel 15 could be achieved in this section. Thus the seal and thereby the outflow of blood from the outflow channel 15 to the environment could be prevented additionally.

In FIG. 9 to 12 a further and possibly independent embodiment of the removal unit 1 is shown, wherein for the same parts the same reference numerals and component names are used as in the preceding FIG. 1 to 8. To avoid unnecessary repetition, reference is made to the detailed description in the preceding FIG. 1 to 8.

In the example embodiment shown here only the insert 20 is shown without the main body 16 receiving the latter and the cannula 3. Said additional insert 20 is designed in a similar way, as described above in FIG. 5 to 8. Therefore, only the main differences are discussed in detail here.

The insert 20 shown here is inserted into the receiving space 18 of the main body 16, as indicated schematically by dashed lines in FIG. 12. A portion of the outflow channel 15 for forming the air removal device 14 extends inside the insert 20 from the proximal end 24 to the distal end 23 and opens, if provided, into the indication chamber 22. The latter is formed between the distal end 23 of the insert 20 and the receiving space 18 inside the main body 16.

After this a further section of the outflow channel 15 extends in the region of the outer surface 21 between the insert 20 and the receiving space 18 with its inner surface 19. In the region of the outer surface 21 of the insert 20 a plurality of chambers 27 to 29 are arranged behind one another in flow direction. In contrast to the annular groove-shaped embodiment of the individual recesses 39 to 41, as described above in FIG. 5 to 8, the latter are arranged spiral-like behind one another in circumferential direction. As viewed in circumferential direction the individual chambers 27 to 29 or the recesses 39 to 41 forming the latter are additionally limited by a plurality of separating webs 43 arranged distributed around the circumference. Depending on the chosen number and arrangement of the separating webs 43 in this way the number of chambers 27 to 29 can be determined as desired. In the embodiment shown here for example the separating webs 43 are arranged diametrically opposite one another. i.e. offset by 180° to one another.

The first, additional connecting channel 32, which connects the indication chamber 22 to the first chamber 27, is arranged in this example embodiment in the same plane in which the separating webs 43 are also arranged. Depending on the selected gradient and associated number of chambers 27 to 29 between the second, additional connecting channel 33 and the outflow channel 37 a further recess 44 is arranged in the outer surface 21 in this way also to form a flow connection in this area. The individual separating webs 43 with respect to the outer surface 21 have a smaller distance relative to the longitudinal axis 5. By means of this shortened embodiment of the individual separating webs 43 in radial direction between an outer end face 45 of the separating webs 43 and the inner surface 19 of the receiving space 18 of the main body 16 respectively the corresponding end connecting channel 30 or 31 is formed between immediately adjacent, chambers 27 to 29 arranged behind one another.

In contrast to the previously described and represented arrangement of the separating webs 43 it would also be possible to select their number to be different in the direction of the longitudinal extension of the recesses 39 to 41. Thus a higher and/or lower number could be selected relative to the circumferential length of a full circumferential extension of the respective recess 39 to 41.

It should be mentioned that the flow cross-section of the individual connecting channels 30 to 33 can be formed or selected to be different from one another. Thus it would be possible for example for the flow cross-section of the individual connecting channels 30 to 33 to narrow continually from the distal end 23 of the insert 20 to the outflow channel 37. In this way an even better flow seal of the outflow channel 15 can be achieved in this section of the insert 20.

The insert 20 described here can be inserted into a main body 16 with or without an external thread 42, as described above in the first embodiment according to FIG. 1 to 4 or the second embodiment according to FIG. 5 to 8. In this case the corresponding three-dimensional shape of the receiving space 18 is selected as a function of the external shape of the insert 20. The second embodiment according to FIG. 5 to 8 could however also be used with a corresponding design of the receiving space 18 in the first described embodiment with the handling element 38.

It is essential in all of the these embodiments that the use of filter elements is always avoided and that the sealing is performed on a mechanical basis exclusively by a suitable selection, size, arrangement and embodiment of the connecting channels 30 to 33 and the chambers 27 to 29 arranged in between. In this way savings can be made as such medical products are always disposable products which need to be disposed of after use.

As already described above, inside the outflow channel 15 at least in portions thereof a substance or a medium could be introduced or applied therein which causes rapid blood clotting. This occurs mainly in those sections of the outflow channel 15, in order to provide in advance a perfect display of the success of the penetration by the blood entering during the blood removal and on contact to effect the clotting and also accelerate it.

Independently of this or additionally it would also be possible, for example to design the first additional connecting channel 32, which connects the indication chamber 22 to the first chamber 27, with a larger flow cross-section than previously described. In this way it is possible that the removed bodily fluid, in particular the blood, can still enter into the first chamber 27 of the outflow channel 15. Furthermore, in the first chamber 27 here a separate substance or medium can be introduced or applies which performs a rapid pre-test or brief analysis of the entered bodily fluid, in particular blood and the test result is displayed for example optically or by a different signal. In this way depending on the substance or medium used as a result of the shown test result the operating staff can decide prior to performing additional actions whether a sample should be filled into a sample receiving vessel or not. In this way an otherwise subsequent sample removal can be interrupted briefly or not even started and thus no sample taking tubes need to be wasted unnecessarily.

The optical display can make it possible to come to a conclusion about the test results, for example a color change or representation in one of several prespecified colors. If the entering bodily fluid, in particular blood, comes into contact with the substance or the medium there may be a color display or change in the initial color of the substance or medium. It would also be possible to use other different display methods. The latter only need to provide a quick pre-test result in order to assist with a decision relating to the start of the removal process after successful penetration.

The method for the removal of bodily fluid, in particular blood, can always be performed in a similar way with the different embodiments of the removal unit 1 as follows.

With a removal unit 1 which has been prepared correctly, and in particular is preferably sterile, firstly a protective element covering the distal cannula segment 6, in particular a protective cap is removed in order to release the insertion end of the cannula 3 facing the patient. Afterwards the distal cannula segment 6 is pierced into the intended body part and thus access is obtained to the bodily fluid to be taken. By means of this piercing process with a correct penetration the bodily fluid to be taken, in the present case blood, will flow through the removal channel 8 of the cannula 3 up to its proximal cannula segment 7. On the basis of this inflow the air contained in the removal channel 8 is removed or extracted from the latter through the outflow channel 15 to the outer environment. Since also the proximal cannula segment 7 is completely covered by the elastically deformable sleeve 4, there can also be no outflow of air through said sealed sleeve 4.

If the sleeve 4 is already made from a translucent or transparent material, in this area the success of the penetration can be determined by eye by the operating staff. If a transparent material is not to be used the bodily fluid, namely the blood, flows along the outside of the cannula 3 through the outflow channel 15 formed in between up to the insert 20 and further through the latter up to the indication chamber 22. In said indication chamber 22 the success of the penetration is now optically visible. The outflow of air through the additional adjoining section of the outflow channel 15 is performed through the previously described connecting channels 30 to 33 and the chambers 27 to 29 arranged in between. The last chamber 29 can then be in flow connection via the annular channel 36 and the outflow channel 37 with the external environment. Determined by the corresponding choice of cross section for the connecting channels 30 to 33 and the chambers 27 to 29 in between, in this section of the outflow channel 15 there is preferably only an outflow of fair, and the passage of bodily fluid, in particular blood, is prevented in the region of the connecting channels 30 to 33.

This previously described method or procedure for the removal of bodily fluids, in particular blood, can also represent an independent objective of the invention and can be achieved by said process according to the invention.

The exemplary embodiments show possible embodiment variants of the removal unit 1, whereby it should be noted at this point that the invention is not restricted to the embodiment variants shown in particular, but rather various different combinations of the individual embodiment variants are also possible and this variability, due to the teaching on technical procedure, lies within the ability of a person skilled in the art in this technical field.

Furthermore, individual features or combinations of features from the shown and described different example embodiments can in themselves represent independent solutions according to the invention.

The problem addressed by the independent solutions according to the invention can be taken from the description.

All of the details relating to value ranges in the present description are defined such that the latter include any and all part ranges, e.g. a range of 1 to 10 means that all part ranges, starting from the lower limit of 1 to the upper limit 10 are included, i.e. the whole part range beginning with a lower limit of 1 or above and ending at an upper limit of 10 or less. e.g. 1 to 1.7, or 3.2 to 8.1 or 5.5 to 10.

Mainly the individual embodiments shown in FIGS. 1 to 4, 5 to 8 and 9 to 12 can form the subject matter of independent solutions according to the invention. The objectives and solutions according to the invention relating thereto can be taken from the detailed descriptions of these figures.

Finally, as a point of formality, it should be noted that for a better understanding of the structure of the removal unit 1, the latter and its components have not been represented true to scale in part and/or have been enlarged and/or reduced in size.

### LIST OF REFERENCE NUMERALS

| | |
|---|---|
| 1 | removal unit |
| 2 | needle carrier |
| 3 | cannula |
| 4 | sleeve |
| 5 | longitudinal axis |
| 6 | distal cannula segment |
| 7 | proximal cannula segment |
| 8 | removal channel |
| 9 | distal needle carrier end |
| 10 | proximal needle carrier end |
| 11 | distal sleeve end |
| 12 | proximal sleeve end |
| 13 | accommodating chamber |
| 14 | air removal device |
| 15 | outflow channel |
| 16 | main body |
| 17 | proximal end section |
| 18 | receiving space |
| 19 | inner surface |
| 20 | insert |
| 21 | outer surface |
| 22 | indication chamber |
| 23 | distal end |
| 24 | proximal end |
| 25 | arrow |
| 26 | arrow |
| 27 | chamber |
| 28 | chamber |
| 29 | chamber |
| 30 | connecting channel |
| 31 | connecting channel |
| 32 | connecting channel |
| 33 | connecting channel |
| 34 | ring base |
| 35 | end wall |
| 36 | annular channel |
| 37 | outflow channel |
| 38 | handling element |
| 39 | recess |
| 40 | recess |
| 41 | recess |
| 42 | external thread |
| 43 | separating web |
| 44 | recess |
| 45 | end face |

1: Medical removal unit (1) for bodily fluids, in particular blood, comprising a needle carrier (2), which needle carrier (2) has a distal needle carrier end (9) and a proximal needle carrier end (10), at least one cannula (3), which cannula (3) comprises a distal cannula segment (6) for sticking into the body and a proximal cannula segment (7) for piercing a closing body of a collecting container, a removal channel (8), which removal channel (8) connects the distal cannula segment (6) to the proximal cannula segment (7), a hose-like elastically deformable and pierceable sleeve (4), which sleeve (4) comprises a closed proximal sleeve end (12) and an open distal sleeve end (11), wherein the distal sleeve end (11) of the sleeve (4) is secured to the proximal needle carrier end (10) of the needle carrier (2) and surrounds the proximal cannula segment (7) in a sealing manner forming an accommodating chamber (13), and with an air removal device (14), which air removal device (14) connects the accommodating chamber (13) for the outflow of air from the accommodating chamber (13) to the outer environment, wherein the air removal device (14) is formed by at least one outflow channel (15) arranged or formed in the needle carrier (2), wherein a portion of the outflow channel (15) comprises a plurality of chambers (27, 28, 29) arranged spaced apart from one another and behind one another in outflow direction and at least one connecting channel (30, 31) connecting chambers (27, 28, 29) arranged directly adjacent to one another in outflow direction and wherein the connecting channel (30, 31) has a flow cross-section which allows the through-flow of air, but prevents or completely blocks the passage of bodily fluid, in particular blood, at least for the most part.

2: The removal unit (1) as claimed in claim 1, wherein the connecting channels (30, 31) are arranged to be offset relative to one another in peripheral direction, in particular diametrically to one another, between chambers (27, 28, 29) arranged directly after one another.

3: The removal unit (1) as claimed in claim 1, wherein a receiving space formed by each of the chambers (27, 28, 29) is designed to be multiple times greater than the volume formed by the connecting channel (30, 31).

4: The removal unit (1) as claimed in claim 1, wherein the needle carrier (2) comprises a main body (16) with a receiving space (18) formed at its proximal end section (17) with an inner surface (19) delimiting the receiving space (18) and an insert (20) received in the receiving space (18) and held on the main body (16) with an outer surface (21) facing the inner surface (19).

5: The removal unit (1) as claimed in claim 4, wherein the chambers (27, 28, 29) are arranged or formed between the inner surface (19) of the receiving space (18) of the main body (16) and the outer surface (21) of the insert (20).

6: The removal unit (1) as claimed in claim 4, wherein the chambers (27, 28, 29) are formed respectively by recesses (39, 40, 41) which are recessed in the outer surface (21) of the insert (20).

7: The removal unit (1) as claimed in claim 4, wherein the recesses (39, 40, 41) forming the chambers (27, 28, 29) are formed respectively by annular grooves arranged spaced apart from one another in the direction of a longitudinal axis (5).

8: The removal unit (1) as claimed in claim 4, wherein the recesses (39, 40, 41) forming the chambers (27, 28, 29) are arranged behind one another in circumferential direction and are separated from one another spiral-like respectively by separating webs (43).

9: The removal unit (1) as claimed in claim 4, wherein as viewed in axial cross-section the inner surface (19) of the receiving space (18) of the main body (16) is designed to widen step-like in the direction of its proximal end section (17).

10: The removal unit (1) as claimed in claim 4, wherein as viewed in axial cross-section the outer surface (21) of the insert (20) is designed to widen step-like in the direction of its proximal end (24).

11: The removal unit (1) as claimed in claim 1, wherein the chambers (27, 28, 29) are designed respectively to run continuously around the circumference.

12: The removal unit (1) as claimed in claim 1, wherein the needle carrier (2), in particular its main body (16), is connected securely to the cannula (3).

13: The removal unit (1) as claimed in claim 1, wherein the outflow channel (15) is arranged or designed to run in outflow direction immediately after the accommodating chamber (13) between the cannula (3) and the needle carrier (2), in particular its insert (20).

14: The removal unit (1) as claimed in claim 1, wherein inside the outflow channel (15) an indication chamber (22) is provided for displaying the success of the penetration for the subsequent removal of blood.

15: The removal unit (1) as claimed in claim 14, wherein the indication chamber (22) is formed by a portion of the receiving space (18) of the main body (16) and a distal end (23) of the insert (20).

16: The removal unit (1) as claimed in claim 1, wherein the sleeve (4) is made at least partly from a translucent or transparent material.

17: The removal unit (1) as claimed in claim 1, wherein the needle carrier (2), in particular its main body (16), is made at least partly from a translucent or transparent material.

18: The removal unit (1) as claimed in claim 1, wherein the removal channel (8) is designed to be continuous between the distal cannula segment (6) and the proximal cannula segment (7) inside the cannula (3).

19: The removal unit (1) as claimed in claim 1, wherein the needle carrier (2), in particular its main body (16), is provided with an external thread (42), wherein the needle carrier (2) can be connected to a distal face end of a handling element (38) for receiving a blood removal tube.

20: The removal unit (1) as claimed in claim 1, wherein the needle carrier (2), in particular its main body (16), is connected to a distal face end of a handling element (38) for receiving a blood removal tube.
